(19) United States
(12) Patent Application Publication
(10) Pub. No.: US 2006/0173267 A1
(43) Pub. Date: Aug. 3, 2006

(54) Method and device for administering health care remotely

(75) Inventors: Chun Hsien Chiang, (US); Craig Anthony Miller, (US)

Correspondence Address: Craig Miller, 642 Via Umbroso, San Clemente, CA, 92672, US

(73) Assignee: Aprius Medical Service, Inc.

(21) Appl. No.: 11/022,566

(22) Filed: Dec. 27, 2004

(51) Int. Cl. A61B 5/05

(52) U.S. Cl. 600/407

(57) ABSTRACT

The video medicine method and system disclosed includes at least one remote center configured to examine a patient, communicate with a diagnostic center enabling a doctor to provide medical care via video conference.

The present invention relates generally to methods, hardware and software for communicating medical services through electronic means. More particularly, the present invention relates more particularly to a method of providing medical services electronically, so as to enable a broader availability of a doctor.

### BACKGROUND

The present invention relates to systems and methods for examining a patient, and more particularly, to the methods of using communications diagnosis of a patient in a remote location.

A number of systems have been developed for use in medical examinations, some of which can be applied in a telemedicine context, i.e., in which data or results from the examination are transmitted over an electronic or other communication link and saved in a central electronic medical record database so that a physician or other medical worker need not be in the same location as the patient. For example, telemedicine systems for performing vision testing and eye examination are described in U.S. Pat. Nos. 4,761,071; 5,617,157; 5,694,199; 5,912,720; 5,943,116; 5,993,001; 6,003,991; 6,022,315; 6,027,217; and 6,033,076. These patents, however, only use either a camera or a video/audio feedback mechanism as diagnostic equipment. Thus, the patents do not disclose systems or methods for collecting a patient's, critical vital signs like blood pressure and heart rate such as to enable a full examination of a patient and enable real-time video communication to multiple remote locations.

As the costs of healthcare continually rise there are more patients that can no longer afford medical health insurance. Furthermore, companies are reducing their participation in medical coverage programs or all together are no longer offering medical insurance for their employees. The reduction in medical coverage forces patients to use emergency medical facilities to treat common illnesses or even worse to not seek medical attention. Accordingly, there is a need in the art for a method that integrates the operation of several optical devices and processes to allow an examination to be conducted on a patient from a remote location.

### BRIEF SUMMARY

The present invention provides a unique method of combining telemedicine systems with traditional methods of providing healthcare that overcome issues with both telemedicine and traditional healthcare. The method includes a diagnostic center for analyzing the information collected, a remote center for collecting the information to be examined, and may include a medical staff which resides at the remote center to insure the accuracy of the information retrieved and a communication system. The diagnostic center may further include a central electronic database for saving and maintaining records corresponding to the collected information and an exam console for enabling a diagnosis based on the collected information.

The communications system in the method and system of the invention includes a video link which enables the method of video conferencing with a doctor located at the diagnostic center. The communication link enables and facilitates the video conferencing to commence independent of the doctor's or patient's location.

Still further, it is another object of the present invention to achieve the foregoing objectives by enabling the communication of examination data collected by medical staff at the remote center via a communications link capable of transmitting large amounts of data and video.

With these and other objects, advantage and features of the invention that may become hereinafter apparent, the nature of the invention may be more clearly understood by reference to the following detailed description of the invention, the claims and to the accompanying drawings.

### BRIEF DESCRIPTION OF THE DRAWINGS

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

FIG. 1 is a schematic view of the remote center as described in the proffered embodiments; and

FIG. 2 is a flowchart representation of a timeline of events of one process as described in the preferred embodiment.

### DETAILED DESCRIPTION OF THE PREFERRED EMBODIMENTS

The present invention incorporates the use of commercially available communication services and devices to enable a method of delivering healthcare from and to remote locations. The equipment is integrated via hardware and software into a comprehensive medical examination system. This system is designed to be operated with assistance from medical staff to ensure accurate results but also may be designed to be operated by the patient or even fully automated. The method generally comprised of three sub-systems, the diagnostic center, the remote center which may include a medical assistant and a communication system. The remote center is the location wherein the patient interacts with diagnostic tools for testing, or where medical staff administers the testing on the patient, to gather the relevant information. The remote center is generally remote from the diagnostic center, however it can be appreciated that the remoteness of the location may comprise the distance of a room, or may include a distance of many thousands of miles. The communication center comprises both the hardware and software that permits the diagnostic center and the remote center to interface and communicate with each other. The diagnostic center comprises the physical location wherein the doctor reviews the examination information communicates with the patient via commercially available video conferencing technologies and makes the patient's diagnosis.

In a preferred embodiment of the invention the communication center is a physical location which enables communication between the diagnostic center and the remote center where the diagnostic center and the remote center are not physically located together and neither is physically located near the communication system. Such that a doctor may use their home or private practice to communicate with the communication system at the same time a remote center may communicate with the communication system. In this embodiment the communication system will smartly synchronize communication between the diagnostic center and the remote center as to also enable video conferencing.

The communication system is a combination of a commercially available medical records program, commercially available hardware which is used to digitally record patient's vital signs, commercially available video conferencing software and costume synchronization software program that smartly synchronizes all three commercially available packages in a method that enables ease of use for doctors.

The purpose of the commercially available medical records program is to store and accurately retrieve patient records in a timely manner such that a doctor can view the prior medical history of a patient. The purpose of the commercially available hardware is to digitally record patients vitals {blood pressure, pulse and lung health} such that a medical assistant can locally administer these tests and obtain recordings or a patient can self administer these tests and obtain digital records. The purpose of the video conferencing software is to allow the patient and doctor to interact in real time thus allowing the more accurate communication and enabling the doctor a human touch interaction with the patient. The purpose of the communication system is to synchronize the doctor who may be communicating remotely with the patient who may also be communicating remotely thus enabling video conferencing while smartly obtaining, archiving and displaying the diagnostic information digitally recorded by the hardware and the medical records information provided by the medical records program. The communication system may also independently drive the medical records program, the hardware to digitally record patient's vitals, the diagnostic center, the remote center and the video conferencing system.

In a preferred embodiment of the present invention the method for enabling a accurate telemedicine uses a communication system which manages communication between the communication system drives the equipment that captures the diagnostic information created by the digital recording hardware with no regard to whom triggers the process medical staff, a patient or a third party. In this embodiment the data is captured at a remote center where the patient is present. The captured data is then transmitted to the communication system via electronic means. The communication system will smartly assign a label to the information and then sends the information to a central electronic medical records database in a manner that can be recalled via the use of the smartly assigned label. The captured data is not restricted to multiple types of data including, patient's vitals, images of patient's ears, nose and throat, images of the patient's skin or hair, and general information about the patient such as name, age and past medical history. The communication system will smartly assign a label to the information sent to the medical records database to enable the communication system to later retrieve the specific data being stored for a specific patient. Once the information is collected and smartly recorded the patient will await at a commercially available video conferencing station at which time a trigger is used to notify the communication system that the patient is ready for their video consultation with a doctor. Furthermore, in the present embodiment a doctor, when available, is enabled to remotely login into a diagnostic system which is a part of the communication system, where the doctor is smartly displayed all diagnostic information including but not limited to images of patient, medical history of patient, and any other relevant information for conducting a medical examination. When triggered the diagnostic system will communicate to the communication system that the doctor is ready to communicate with the patient. At that time the communication system will initiate a synchronization process enabling the doctor and the patient to communicate via video conference.

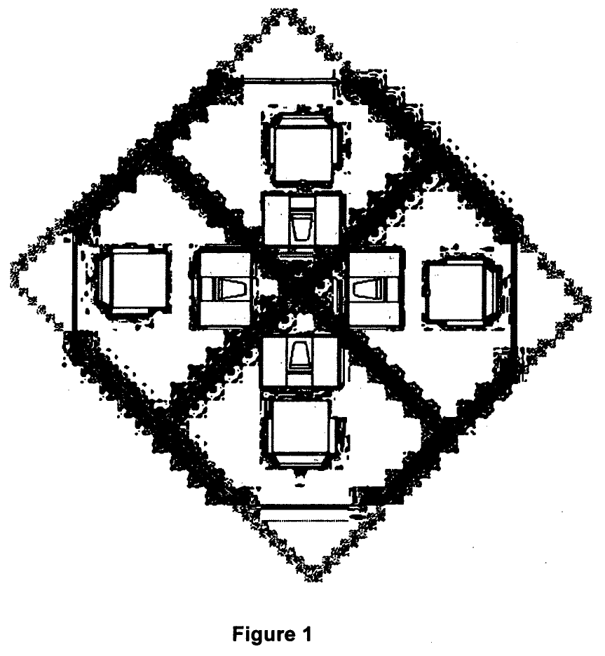

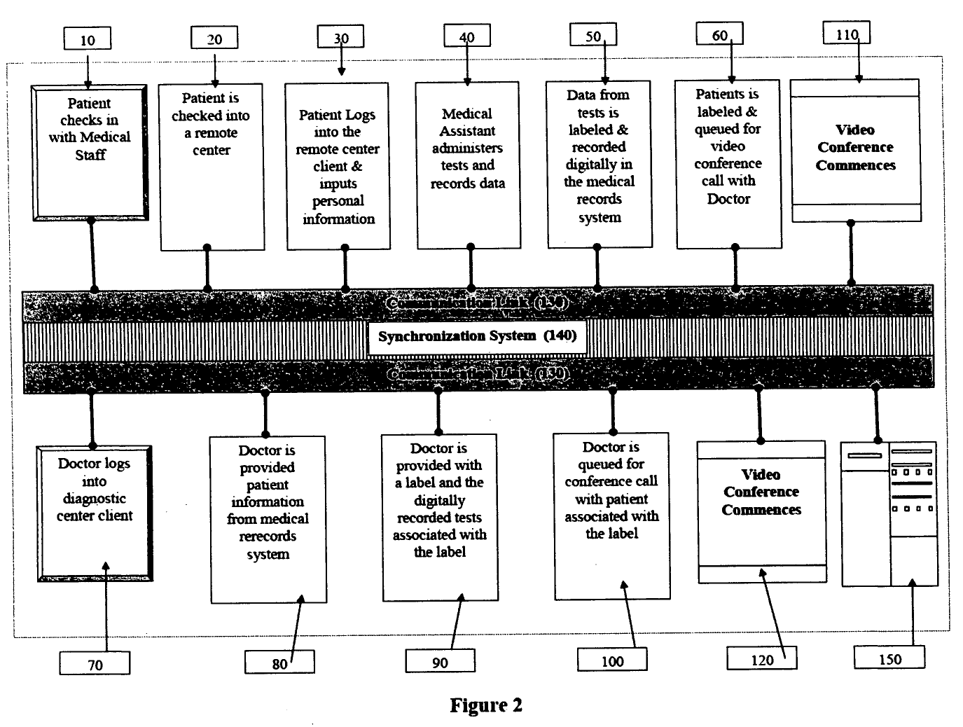

In the detail description of the preferred embodiment as shown in the figures and in the current embodiment a remote station (10) is used and shown in FIG. 2. The process as described in this embodiment is illustrated in FIG. 2 where a patient checks in with a medical staff located near the remote center (20). Once the medical patient is recognized by the medical staff they are directed to check into the remote center (20). The patient then precedes to the next step of which is to log-in to the remote center client (30) and input personal information which may include personal contact information, medical history, family medical history, any known allergies or aversions to drugs, etc. In the following steps as illustrated in FIG. 1 a medical assistant administers tests and records data resulting from the tests (40). It is conceivable to use devices and technology known in the art that the patient may be able to administer the tests performed by a medical assistant. Furthermore, it is conceivable with the use of devices and technology known in the art for an automated medical system to perform tests and record data without the need of a medical assistant. As data is collected and digitally recorded the communication link (130) is communicating the data to the communication system (150) which includes at least one server for computing and storing the recorded data in a medical records system. The next process in the system is performed by the communication system (150) which smartly labels the digitally recorded data that references a specific patient the digitally recorded data relates to. The patient is also assigned a label (60) which associates the patient with the patient's digitally recorded data in the medical record system and placed in a queue for remote video conference (110) which is managed by the synchronization system (140). At the same time or at a different time a doctor logs onto a diagnostic center (70) which may be located at the same location as the communication system (150) or may be located at a remote and different location. The doctor is smartly configured and is provided patient information with a specific label (60) which is used to identify the patient and the patients records (50). By use of the label, the doctor is provided the data from tests that were digitally recorded and stored in the medical records system which is associated with a specific patient by way of the communication link (130). The doctor is queued for a diagnostic video conferencing call (120) with the patient at remote video conferencing call (110). Once the doctor and patient are queued for a video conferencing call the synchronization system (140) will synchronize any data between the remote video conferences (110) and the diagnostic video conference (120) and trigger the video conferencing system to commence the video conference. Upon completing the video conference the data is digitally stored including any additional data added by the patient or doctor during the video conferencing call. Furthermore, a picture of the patient is digitally stored and the video may or may not be digitally stored for future reference. In the present embodiment it is easily conceivable to enable a questionnaire to be electronically displayed at the remote center and/or the diagnostic center which would be used for feedback to improve the methods and system.

The process described above enables a doctor to conduct a video conference with a patient thus reducing costs associated with healthcare and health insurance. More specifically the process described above allows doctors a more flexible schedule and to be in a remote location which enables more a lower cost for delivering healthcare while offering a broader range in schedule. Furthermore, the reduction in costs enables patients that do not have healthcare insurance access to a doctor. The more readily available access to a doctor will allow patients to communicate and obtain medical care before a medical issue develops into a medical emergency which needs more medical attention at a higher costs and may be life threatening.

While this invention has been described in conjunction with the specific embodiments outlined above, it is evident that many alternatives, modifications and variations are apparent to those skilled in the art. Accordingly, the preferred embodiments of the invention as set forth above are intended to be illustrative and not limiting. Various changes may be made without departing from the spirit and scope of the invention.

What is claimed is:

1. A telemedicine system comprising:
at least one remote center including, a plurality of hardware configured to obtain patient's information and vitals;
a diagnostic center for analyzing the collected information; and
a communications system which labels the collected information and stores the information into a medical record database;
wherein the communication system enables the real-time display of medical record information relating to a specific label; and
for enabling communication and carrying the collected information between the remote center;
wherein the communication system comprises, a record database for maintaining label information; and
a synchronization system that can synchronize the diagnostic center, the remote center and all information related to the label thus enabling a real-time video conferencing session.

2. The system of claim 1, wherein the communications link is an Internet connection.

3. The system of claim 1, further comprising computer means for analyzing the collected data and diagnosing conditions.

4. The system of claim 1, wherein the information comprises at least one of the following: Vital signs, pulse, lung health, patient medical history, blood pressure.

5. The system of claim 1, wherein a patient being examined in one of the at least one remote exam modules confers with a doctor in the diagnostic center via real-time videoconferencing.

6. The system of claim 1, wherein the at least one remote center includes a display unit for displaying a questionnaire to a patient for collection of patient history data.

7. The system of claim 6, wherein the display unit is provided with a touch-screen that allows the patient to enter responses to the questionnaire.

8. The system of claim 6, wherein the patient's case history is posted on a web site for access by a patient.

9. A telemedicine system comprising:
at least one remote center including, a plurality of hardware configured to obtain patient's information;
a diagnostic center for analyzing the collected information; and
